(19) United States
(12) Patent Application Publication
(10) Pub. No.: US 2004/0015101 A1
(43) Pub. Date: Jan. 22, 2004

(54) Rapid non-invasive method for differential acute cardiovascular disease diagnosis

(76) Inventors: Yoram Rubin, Haifa (IL); Shai Nimri, Kibbutz Nir David (IL); Nitsa Galili-Nachshon, D.N. Gilboa (IL); Sari Alon, Yuvalim (IL); Inbal Ben-Tzvi Tzchori, Yehoshua (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/341,527

(22) Filed: Jan. 13, 2003

Related U.S. Application Data
(63) Continuation of application No. PCT/IL01/00640, filed on Jul. 12, 2001.

(30) Foreign Application Priority Data
Jul. 13, 2000 (IL) 137307

(51) Int. Cl. A61B 5/00

(52) U.S. Cl. 600/584

(57) ABSTRACT

The invention relates to a non-invasive method for the diagnosis of cardiovascular disease in a subject, comprising the steps of measuring in a urine sample the concentrations of one or more thromboxanes, the conductivity, and the concentration of at least one additional marker of cardiovascular disease, which marker can be chosen from apolipoprotein (a), conjugated dienes and lipid peroxides. Diagnosing the presence of cardiovascular disease in said subject is carried out by comparison of the analytical results with pre-determined reference values. The invention further relates to a kit for the rapid diagnosis of cardiovascular diseases.

### RELATED APPLICATION

This application is a continuation of International Application PCT/IL01/00640 filed Jul. 12, 2001 designating the United States of America, for which priority is claimed under 35 USC 120.

### BACKGROUND OF THE INVENTION

1. Field of the Invention

The present invention is concerned with a method for diagnosing cardiovascular disease by the assay of urinary thromboxanes and at least one additional marker of cardiovascular disease, wherein the marker is chosen from urinary apolipoprotein (a), conjugated dienes, or lipid peroxides. The method disclosed hereinbelow is particularly useful for the rapid differential diagnosis of cardiovascular disease.

2. Prior Art

The group of diseases affecting the heart and blood vessels is one of the leading causes of morbidity and mortality. In particular, Acute Coronary Syndrome (ACS) is a leading cause of death in the Western world. While the group of cardiovascular disease taken as a whole consists of a large number of different disease entities, each with it own specific pathogenetic factors, a common element among many of the most prevalent cardiovascular conditions is the formation of athersclerotic plaque, with all its varied biochemical and pathophysiological consequences.

On a worldwide scale, more than 70 million people present at hospitals and other primary health care providers complaining of chest pain each year. In the United States alone, over six million people present with chest pain each year, a statistic that is reflected in the fact that cardiovascular disease accounts for fully one quarter of the current annual health expenditure in the US.

Since the effectiveness of treatment falls exponentially from the time of a myocardial event, the ability to rapidly and accurately diagnose cardiovascular pathology, and thereby commence appropriate treatment at a much earlier stage, is critical in reducing the number of deaths from heart disease.

An additional medical benefit to be derived from improved diagnostic technology screening is the capability to detect patients at risk of developing atherosclerotic lesions and subsequent cardiovascular (and cerebrovascular) pathology. This is of obvious benefit to the development of reliable strategies for the prevention of serious cardiovascular disease.

Finally, the development of early and accurate diagnostic tests will enable health services to reduce the number of unnecessary hospital stays and expensive tests that are administered, providing significant cost savings. Currently, the total annual cost of testing patients for ACS, according to the American College of Cardiology, is estimated to be about $6 billion.

The thromboxanes are compounds derived from prostaglandin endoperoxides that cause platelet aggregation, arterial contraction and many other biological effects. One such compound, thromboxane A₂, a highly unstable biologically active bicyclic oxitane-oxane compound, displays very potent vasoconstricting and platelet aggregating properties. thromboxane A₂ has been found to play a crucial role in atherothrombotic disorders, and increased synthesis therof has been found to occur immediately following events such as unstable angina and acute myocardial infarction [Fitzgerald, D. J. et al. (1986) N. Engl. J. Med. 315: 983-989]. As mentioned above, thromboxane A₂ is very unstable, and is rapidly converted to stable metabolites such as 11-dehydrothromboxane B₂ and 2,3, di-northromboxane B₂ (collectively referred to hereinbelow as “thromboxane B₂”), which are excreted in the urine.

Apolipoprotein (a) (hereinafter abbreviated as Apo(a)) is a glycoprotein having a carbohydrate concentration of approximately 29% (w/w), and a characteristic protein structure consisting of numerous kringle-IV repeats, one kringle-V unit, and a protease domain [Kostner, K. M. et al. (1997) Atherosclerosis 129: 103-110].

Although a full picture of the physiological and pathological significance of Apo(a) is yet to emerge, an association with cardiovascular disease has been reported. One report, for example [Kostner, K. M. et al. (1997) CardioSource 129: 103-110] describes the fact that patients suffering from coronary artery disease excrete higher amounts of Apo(a) fragments into their urine than do control subjects.

It is widely accepted that lipid peroxidation plays a central role in the development of cardiovascular diseases and that low-density lipoprotein (LDL) oxidation is an indication of early atherosclerosis. General markers of LDL oxidation are conjugated dienes (CD) and lipid peroxides (PD) which can be determined quantitatively [Aviram M. et al. (2001) Methods in Enzymology 235:244-248].

It is a purpose of this invention to provide an assay for the accurate diagnosis of cardiovascular conditions. The terms cardiovascular conditions and cardiovascular diseases as used herein are to be taken to mean pathological conditions of the heart or blood vessels, including atherosclerotic conditions and pathological thrombogenic conditions.

It is another purpose of the invention to provide a diagnostic assay that may be used as an early-warning, first window test.

A further object of the invention is to provide a diagnostic assay that is simple to use and which yields rapid results.

It is yet another object of this invention to provide a diagnostic assay that may assist in the differential diagnosis of acute cardiovascular conditions.

Other objects and advantages of the invention will become apparent as the description proceeds.

### SUMMARY OF THE INVENTION

It has now been found that the information derived from the determination of the concentrations of thromboxanes and at least one additional marker of cardiovascular disease, chosen from apolipoprotein (a), conjugated dienes and lipid peroxides, in urine samples may be used as a powerful diagnostic tool in patients suspected of suffering from cardiovascular disease, in particular, acute cardiovascular syndrome. Unexpectedly, the combination of the determination of at least two of the above-mentioned analytes provides much greater diagnostic information than the measurement of each analyte alone, particularly in relation to the ability of this multi-measurement method to provide differential diagnostic data.

The invention is primarily directed to a method for the diagnosis of cardiovascular disease in a subject comprising the steps of:

a) obtaining a sample of urine from said subject;

b) measuring the concentrations of one or more thromboxanes selected from the group consisting of thromboxane B₂ 11-dehydrothromboxane B₂, 2,3-di-northromboxane B₂, and mixtures thereof, in said urine sample;

c) measuring the concentration of at least one additional marker of cardiovascular disease, chosen from Apo(a) and/or isoforms thereof, conjugated dienes and lipid peroxides in said urine sample;

d) diagnosing the presence of cardiovascular disease in said subject by comparison of the results obtained in steps b) and c) with a pre-determined reference value;

wherein steps b) and c) may be performed either consecutively in any order, or simultaneously.

In one preferred embodiment of the invention, the method further comprises measuring the electrical conductivity of the urine sample, wherein the thromboxane concentrations are expressed as the ratio of the measured thromboxane concentration to said electrical conductivity.

In a preferred embodiment of the method of the invention, the thromboxane measured is thromboxane B₂. In one preferred embodiment of the invention, the concentrations of one or more thromboxanes and of Apo(a) are measured using an amperometric assay. A preferred amperometric assay for use in the method of the present invention is disclosed in co-pending Israeli Patent Application No. 132410.

In another preferred embodiment of the invention, the concentrations of the one or more thromboxanes and of Apo(a) are measured using a biosensor device. Many different types of biosensor device may be used to perform these measurements. In one preferred embodiment, the biosensor device is a fluorescence-based biosensor device. In another preferred embodiment of the invention, the biosensor device is a spectrophotometric-based biosensor device. In a further preferred embodiment of the method of the invention, the biosensor device is a semiconductor-based device.

In another preferred embodiment of the invention, the thromboxane and/or Apo(a) concentrations are measured using a immunoassay. Although many different types of immunoassay may be used, in a preferred embodiment, the immunoassay is an enzymeimmunoassay.

In yet another embodiment of the invention, the thromboxane and/or Apo(a) concentrations are measured using an immunoturbidimetric assay.

In another preferred embodiment of the invention, the thromboxane and/or Apo(a) concentrations are measured using an antibody library phage display technique.

In a further preferred embodiment, the thromboxane and/or Apo(a) concentrations are measured using an aptamer-based assay.

In a still further preferred embodiment of the method of the invention, the thromboxane and Apo(a) concentrations are measured using a dipstick-type assay.

In another preferred embodiment of the invention, the concentrations of one or more thromboxanes and of conjugated dienes are measured, wherein the dienes are determined using a spectrophotometric assay.

In still another preferred embodiment of the invention, the concentrations of one or more thromboxanes and of lipid peroxides are measured, wherein the peroxides are determined using either a spectrophotometric assay or a redox titration, preferably iodometric titration.

The present invention also encompasses a kit for the rapid diagnosis of cardiovascular disease comprising:

a) a receptacle for collection of urine samples;

b) means for measuring the urinary concentration of one or more thromboxanes selected from the group consisting of thromboxane B₂, 11-dehydrothromboxane B₂, 2,3-di-northromboxane B₂, and mixtures thereof;

c) means for measuring the urinary concentration of any one of Apo(a), conjugated dienes and lipid peroxides;

d) a reference chart for interpretation of the results obtained in b) and c) and for assessing the diagnostic significance of said results; and

e) manufacturer's instructions for use of said kit.

The kit according to the invention comprises a receptacle with tubes enabling the measurement of some of the markers of cardiovascular diseases, recited above, directly in said tubes. The measurement can comprise spectrophotometry, turbidimetry, immunoassays, or titrations. Suitable and preferred means for measuring said concentrations are dipstick type devices.

In one preferred embodiment of the invention, the color-forming reactions of a spectrophotometric assay, that is a part of the kit, can be performed directly in said tubes, which are provided with stoppers. In another preferred embodiment, the tubes are transparent and for use with a spectrophotometer. The most preferred tubes are adopted for direct reading of absorbance in a spectrophotometric assay.

In other preferred embodiments of the invention, the kits comprise reagents for determination of one or more of the markers of cardiovascular diseases in urine using a spectrophotometric assay or/and an immunoassay.

A kit according to the invention preferably comprises also means for measuring the conductivity of said urine samples. A preferred kit comprises means for measuring the conductivity of said urine sample. In another preferred embodiment of the invention, the kit comprises a conductometric electrode that is adopted for measuring the conductivity of said urine sample directly in said tube.

All the above and other characteristics and advantages of the invention will be further understood from the following illustrative and non-limiting examples of preferred embodiments thereof.

### DETAILED DESCRIPTION OF PREFERRED EMBODIMENTS

The urinary concentrations of the analytes measured in the method of the present invention may be obtained by the use of any suitable quantitative or semi-quantitative analytical technique. Such techniques for thromboxane B₂ compounds, and for Apo(a) and its isoforms include, but are not limited to, enzyme-linked immunoassays (ELISA), radio-immunoassays (RIA), immunoturbidimetric assays, amperometric assays, dipstick-type assays and measurements using semiconductor-based devices. These techniques are all extensively described in the art, and well known to the skilled artisan in this field. In the case of dipstick-type assays, antibodies and reagents suitable for the quantitative or semi-quantitative detection of both Apo(a) and thromboxane B₂ would be incorporated onto the same dipstick, and appropriate color charts would be provided for the interpretation of data thus obtained. Similarly, biosensor devices could be used as the measurement apparatus for determining the concentrations of the two analytes involved in the method of the present invention. Examples of suitable biosensors include fluorescence-based devices, spectrophotometric devices and semi-conductor based devices. In the latter case, separate channels of the device would be used for the separate determination of the concentrations of Apo(a) and thromboxane B₂, each determination being performed by virtue of the presence of specific antibodies located at spatially-separated locations on the device. Thus, two separate electric currents are produced and analyzed separately, according to one or more interpretive rules (as described in more detail in the following illustrative Example). Additionally and optionally, a third channel might be used for determining the electrical conductivity of the urine sample, as a means of standardizing the thromboxane concentrations (because of their dependence on urinary volume). The measurement of a conductivity-normalized urinary analyte is described in co-pending Israel Patent Application No. 137308. The combined use of conductivity and thromboxane concentration measurements are also described in the following Examples.

In addition to the techniques described hereinabove, the urinary concentrations of the thromboxane and/or Apo(a) analytes may also be measured using an antibody library phage display technique. Many different variations on the basic technology [described in: Burton, D. R. & Barbas, C. F. III (1993) Immunomethods 3:155-163] are known in the art, and may be adapted for use in measuring in conjunction with the method claimed herein.

A further approach for measuring one or both of the analytes of the method of the present invention is the use of aptamer-based assays. Aptamers are nucleic acid molecules that bind specific ligands with high affinity and selectivity [Jayasena, S. D. (1999) Clin. Chem. 45:1628-50]. Although clearly very different from antibodies in terms of structure and means of production, aptamers are beginning to emerge as a class of detection molecules that rival antibodies in both therapeutic and diagnostic applications. They are thus ideally suited for use in the method of the present invention. Many different types of assay have been developed [Osborne, S. E., Masumura, I. & Ellington, A. D. (1997) Curr. Opin. Chem. Biol. 1: 5-9] and may be used for the measurements required by the method of the present invention.

The concentration of conjugated dienes and of lipid peroxides can be determined according to methods reviewed by Aviram [Aviram M. et al. (2001) Methods in Enzymology 235:244-248] or according to their modifications, using spectrophotometry, titrations, TLC, HPLC, GC, etc. The preferred method for determining the concentration of lipid peroxides is iodometry or spectrophotometry, and of conjugated dienes spectrophotometry.

The use of specific biochemical and electrochemical measurement techniques in performing the methods of the present invention, and the interpretation of the results obtained therefrom, are described in the following illustrative and non-limiting Examples.

### EXAMPLES

### Example 1

### Correlation of Thromboxane/Apo(a) Determinations with Clinical Diagnosis

Subjects and Samples:

A group of 44 subjects in the age range 40-70 presenting in the Emergency Room of a large district hospital were randomly selected for this study. Samples of urine were collected from each of the patients before they were subjected to any diagnostic or treatment procedures. These urine samples were immediately frozen and stored at −20° C. for periods of less than one month, prior to being used for the biochemical analyses.

The patients were also asked whether they were currently taking, or had recently been taking, cyclooxygenase inhibitors such as aspirin.

The medical condition of each patient was also assessed 30 days after taking the urine sample, each patient being assigned to one of the following diagnostic groups:

1. MI/MCE (MI=myocardial infarction; MCE=major cardiovascular event)

2. Angina

3. Discharged

In addition, the patients' 30 day outcome was also assessed according to the following two criteria:

1. Any cardiovascular event

2. Free of chest pain

Comparison of the clinical outcome with the result obtained from the biochemical analyses (see below in “Data analysis methods”) was performed, in order to determine the sensitivity and specificity of said biochemical analyses as diagnostic tools.

Biochemical Analyses:

1. Thromboxane B₂ Analysis

The concentrations of thromboxane B₂ in the urine samples were measured using a modification of the Biotrak™ system (Amersham International plc, Little Chalfont, Buckinghamshire, England; code RPN 220). The frozen urine samples were thawed and used directly in the thromboxane assay, without any form of pretreatment.

Briefly, 50 μl of each sample or thromboxane B₂ standard was added in duplicate to the wells of a microtitre plate pre-coated with donkey anti-rabbit IgG. All standard solution dilutions were made in an assay buffer consisting of 0.1M phosphate buffer, pH 7.5 containing 0.9% sodium chloride and 0.1% bovine serum albumin. The same buffer was also used in the preparation of the zero standard (i.e. 0 pg thromboxane B₂) and non-specific binding (i.e. buffer-only) wells. The amount of thromboxane B₂ added to the standard wells varied between 0.5 and 64 pg per well. Next, 50 μl of rabbit anti-thromboxane B₂ antiserum was added to each well (except for the spectrophotometric blank well). Following this, 50 μl of thromboxane B₂-horseradish peroxidase conjugate solution was added to each well (except for the blank well), and the plate incubated with shaking at room temperature for one hour. At the end of this incubation period, the contents of each well were aspirated, and each well washed four times with 400 μl wash buffer (0.01M phosphate buffer, pH 7.5, containing 0.05% Tween 20). Immediately following the final washing step, 150 μl of enzyme substrate (consisting of 3,3′, 5,5′-tetramethylbenzidine and hydrogen peroxide) were added to each well. The plate was then incubated with shaking at room temperature for exactly 15 minutes, to allow development of the colored reaction product. The reaction was stopped by the addition of 100 μl of 1M sulphuric acid into each well. Following thorough mixing, and within 30 minutes of addition of the sulphuric aced, the optical density of each well at 450 nm was determined using a plate reader.

A calibration curve was constructed for the thromboxane B₂ standards by plotting the known thromboxane B amount (x-axis) against the percentage of bound antibody (%B/B₀). The latter parameter was calculated according to the following relationship:

%B/B₀=[(thromboxane standard OD−non-specific binding OD)/(B₀OD−non-specific binding OD)]×100

(wherein each OD reading is the average for duplicate wells).

The sample thromboxane B₂ amounts for the samples were obtained by reading directly from the calibration curve.

2. Conductivity Analysis

The electrical conductivity of each of the urine samples was measured using a CyberScan CON100 conductivity meter (Eutech Instruments Pte Ltd., Singapore). A corrected thromboxane B₂ concentration for each sample tested was obtained by dividing said thromboxane concentration (measured in pg/ml) by the conductivity (measured in mS/cm), either by simple division or by more advanced statistical model.

3. Apo(a) Analysis

Urinary Apo(a) concentrations were measured by use of a commercially-available kit for detection of lipoprotein (a) using the following immunoturbidimetric method (Unimate 3 LPA, Roche Diagnostics, Cat. No. 07 3980 4).

The undiluted urine sample was kept at 2-8° C. prior to the analysis. The sample was then incubated with the following reagents: reagent R (supplied in the kit), rabbit antibodies specific for human lipoprotein (a) (supplied in the kit), lipoprotein (a) standard (LPA T Standard, Roche Diagnostics, Cat. No. 07 51170), lipoprotein (a) control (LPA T Control, Roche Diagnostics, Cat. No. 07 51197) and NaCl solution 154 mmol/L (0.9%). The precipitate formed following 10 minutes incubation was determined turbidimetrically using a chemical analyzer (Cobas Mira, COBAS instruments), and converted to protein concentration by the use of a calibration curve created from results obtained with the specific lipoprotein (a) standard solution.

Data Analysis Methods:

Cut-Off Determination:

The cut-off indicates a value which dictates if the patient condition is pathological or normal. Cut-off was determined according to Receiver Operating characteristic Curves (ROC), which is a plot of the sensitivity (or the true positive values) vs. the false positive values. This analysis optimizes the correlation between the test results and the clinical outcome.

The cut-off values are the reference values used in the method of the invention. Preferably, such reference values are based on results of diagnostic tests of large groups of patients.

The results of the various analyses described hereinabove were collected and analyzed according to the following three interpretive ‘rules’.

Rule 1 is based on measuring thromboxane B₂ concentrations and conductivity, and transforming a thromboxane/conductivity ratio to its natural logarithm, wherein a positive result (i.e. the presence of cardiovascular disease) is indicated by a natural logarithm-transformed ratio greater than the cutoff value of 3.2 for patients not taking cyclooxygenase inhibiting drugs (e.g. aspirin), or greater than the cutoff value of 2.7 for patients that are taking or have recently taken such drugs.

Rule 2 is based on measuring Apo(a) concentrations alone, wherein a positive result (i.e. the presence of cardiovascular disease) is indicated by an Apo(a) concentration equal to or greater than the cutoff value 20 mg/dl

Rule 3 is based on measuring thromboxane B₂ concentrations, conductivity and Apo(a) concentrations, wherein a positive result (i.e. the presence of cardiovascular disease) is indicated by a thromboxane/conductivity ratio greater than the cut-off points given in Rule 1 and an Apo(a) concentration greater than the cutoff value of 20 mg/dl.

Following analysis of the data according to the foregoing rules, and tabulation of said data, the sensitivity and specificity of each rule was determined according to the following definitions:

Sensitivity (%)=True positive/(False negative+True positive)×100

Specificity (%)=True negative/(False positive+True negative)×100

Results:

The results comparing the clinical outcome (any cardiovascular event/free of chest pain) with the biochemical results, as interpreted by each of the three aforementioned rules are given in Table I. It may be seen from this table that the sensitivity of Rule 1 (based on thromboxane concentration/conductivity ratio only) was 83.8%, while the specificity obtained with this rule was 30.7%. For Rule 2 (based on Apo(a) measurements alone) the sensitivity dropped to 77.4% while the specificity was reduced to 23%. The best sensitivity results, however, were obtained with Rule 3 (based on a combination of the thromboxane/conductivity results and the Apo(a) measurements). In this case, the sensitivity obtained was 87%, while the specificity was 30.7%.

The predictive strength of the three rules in correctly determining the outcome of patients with major cardiovascular events (including myocardial infarction) and patients with angina, is illustrated in Table II. From this table it may be seen that all rules gave good sensitivity results for predicting major cardiovascular events (Rule 1: 100%; Rule 2: 88.8%; Rule 3: 100%). In the case of angina, however, the rule that yielded the highest sensitivity was Rule 3, that is the rule using both the thromboxane/conductivity data and the Apo(a) measurements (81.8%). The specificity of this rule (30.7%) was the same as rule 1, and higher than that observed in rule 2 (23%).

TABLE I

| 30 Days Outcome | |
|---|---|
| Any | |
| Cardiovascular | Free of Chest |
| Event | Pain |

| | N | % | N | % |
|---|---|---|---|---|
| Rule 1 | | | | |
| 0 | 5 | (16.1%) | 4 | (30.7%) |
| 1 | 26 | (83.8%) | 9 | (69.2%) |
| Rule 2 | | | | |
| 0 | 7 | (22.5%) | 3 | (23.0%) |
| 1 | 24 | (77.4%) | 10 | (76.9%) |
| Rule 3 | | | | |
| 0 | 4 | (12.9%) | 4 | (30.7%) |
| 1 | 27 | (87.0%) | 9 | (69.2%) |

TABLE II

| | | |
|---|---|---|
| 1:MI, MCE | 2:Angina | 4:Discharged |

| | N | % | N | % | N | % |
|---|---|---|---|---|---|---|
| Rule 1 | | | | | | |
| 0 | | | 5 | (22.7%) | 4 | (30.7%) |
| 1 | 9 | (100.0%) | 17 | (77.2%) | 9 | (69.2%) |
| Rule 2 | | | | | | |
| 0 | 1 | (11.1%) | 6 | (27.2%) | 3 | (23.0%) |
| 1 | 8 | (88.8%) | 16 | (72.7%) | 10 | (76.9%) |
| Rule 3 | | | | | | |
| 0 | | | 4 | (18.1%) | 4 | (30.7%) |
| 1 | 9 | (100.0%) | 18 | (81.8%) | 9 | (69.2%) |

### EXAMPLE 2

### Correlation of Thromboxane/Additional Marker Determinations with Clinical Diagnosis

Subjects and Samples:

A group of 27 patients was randomly selected, and samples of their urine were collected in the same manner as in Example 1. Ten patients were free of chest pain, and 17 had a cardiovascular event.

Analytical methods:

1. Tromboxane B₂ was analyzed, and the results were normalized, as described in Example 1. The conductivity was measured as described in Example 1.

2. Determination of Conjugated Dienes

The concentrations of conjugated dienes (CD) in the urine samples were measured using the following spectrophotometric assay. The frozen sample was thawed, vortexed with 2 ml of hexane/isopropanol (3:2), and acidified by vortexing with 1 ml sulfuric acid (1:2000). The upper phase was dried under nitrogen, diluted with hexane and immediately measured at 234 nm. The CD concentration was calculated according to this relationship: nmol CD/ml=OD×10000/27

3. Determination of Lipid Peroxides

The concentrations of lipid peroxides (PD) in the urine samples were measured using a commercially available reagent (CHOD-iodide-Merck, Cat. No. 14106) according to El-Saadani [El-Saadani et al. (1986) J. Lipid Res. 30:627-630]. Shortly, 100 μl sample was vortexed with the color reagent and left 30 minutes in dark. The absorbance at 365 nm was read against the color reagent as the blank, and the concentration of PD was calculated using this relationship: nmol PD/ml=OD/2.46.

Data Analysis Methods:

The results of the various analyses described hereinabove were collected and analyzed as follows. A positive result (i.e. the presence of cardiovascular disease) was indicated by an experimental value greater than a cut-off point, which was varied according to the measured marker. The cut-off value was determined on a probability scale of zero to one, statistically calculated by integrating the following experimental parameters: analytes concentration, urine conductivity and in the case of thromboxane, aspirin intake. The sensitivity and specificity for a desired combination of measurements and certain cut-off values were calculated according to their definitions in Example 1.

The Results:

The results for various models are presented in the following tables, wherein “Test+”and “Test−” mean positive and negative results, respectively, of the biochemical measurement interpretation. “Disease+”and “Disease−” mean presence or absence, respectively, of the disease as found by clinical examination.

Model 1

Conductivity and Thromboxane Were Measured. Cut-Off Value is 0.60

| | Disease+ | Disease− |
|---|---|---|
| Test+ | 12 | 5 |
| Test− | 3 | 7 |

Sensitivity/Specificity
80%/58%

Model 2

Conductivity and Thromboxane Were Measured Together with CD. Cut-Off Value is 0.60

| | Disease+ | Disease− |
|---|---|---|
| Test+ | 14 | 3 |
| Test− | 1 | 9 |

Sensitivity/Specificity
93%/75%

Model 3

Conductivity and Thromboxane Were Measured Together with PD. Cut-Off Value is 0.60.

| | Disease+ | Disease− |
|---|---|---|
| Test+ | 11 | 3 |
| Test− | 6 | 7 |

Sensitivity/Specificity
65%/70%

Model 4

Conductivity and Thromboxane Were Measured Together with Apo(a). Cut-Off Value is 0.60.

| | Disease+ | Disease− |
|---|---|---|
| Test+ | 15 | 2 |
| Test− | 4 | 6 |

Sensitivity/Specificity
79%/75%

### Model 5

Conductivity And Thromboxane Were Measured Together with CD, AD, and Apo(a). Cut-Off Value is 0.60.

| | Disease+ | Disease− |
|---|---|---|
| Test+ | 13 | 4 |
| Test− | 1 | 9 |

Sensitivity/Specificity
93%/69%

It is concluded from the data presented in the above Examples that the use of the multiple biochemical parameters (thromboxane concentration, urine conductivity, Apo(a), CD, and PD), all together or in subgroups, in accordance with interpretive rules described above, significantly increases the accuracy of the test in comparison to using any marker alone, for diagnosing a cardiovascular event.

While specific embodiments of the invention have been described for the purpose of illustration, it will be understood that the invention may be carried out in practice by skilled persons with many modifications, variations and adaptations.

1. A method for the diagnosis of cardiovascular disease in a subject comprising the steps of:
a) obtaining a sample of urine from said subject;
b) measuring the concentrations of one or more thromboxanes selected from the group consisting of thromboxane B₂, 11-dehydrothromboxane B₂, 2,3-di-northromboxane B₂, and mixtures thereof, in said urine sample;
c) measuring the concentration of at least one additional marker of cardiovascular diseases, in said urine sample;
d) measuring the electrical conductivity of the urine sample, and expressing the thromboxane concentrations obtained in step a) as the ratio of the measured thromboxane concentration to said electrical conductivity;
e) diagnosing the presence of cardiovascular disease in said subject by comparison of the results obtained in steps c) and d) with a pre-determined reference value;
wherein steps b) and c) may be performed either consecutively in any order, or simultaneously.

2. A method according to claim 1, wherein the additional marker is chosen from apolipoprotein (a), conjugated dienes, and lipid peroxides.

3. A method according to claim 1, wherein the thromboxane measured is thromboxane B₂.

4. A method according to claim 2, wherein the additional marker is apolipoprotein (a) (Apo(a)).

5. A method according to claim 4, wherein the thromboxane and Apo(a) concentrations are measured using an amperometric assay.

6. A method according to claim 4, wherein the thromboxane and/or Apo(a) concentrations are measured using a biosensor device.

7. A method according to claim 6, wherein the biosensor device is a fluorescence-based biosensor device.

8. A method according to claim 6, wherein the biosensor device is a spectrophotometric-based biosensor device.

9. A method according to claim 6, wherein the biosensor device is a semiconductor-based device.

10. A method according to claim 4, wherein the thromboxane and/or Apo(a) concentrations are measured using an immunoassay.

11. A method according to claim 10, wherein the immunoassay is an enzymeimmunoassay.

12. A method according to claim 4, wherein the thromboxane and/or Apo(a) concentrations are measured using an immunoturbidimetric assay.

13. A method according to claim 4, wherein the thromboxane and/or Apo(a) concentrations are measured using an antibody library phage display technique.

14. A method according to claim 4, wherein the thromboxane and/or Apo(a) concentrations are measured using an aptamer-based assay.

15. A method according to claim 4, wherein the thromboxane and Apo(a) concentrations are measured using a dipstick-type assay.

16. A method according to claim 2, wherein conjugated dienes (CD) serve as the additional marker.

17. A method according to claim 16, wherein the concentration of said CD is measured using a spectrophotometric assay.

18. A method according to claim 2, wherein lipid peroxides (PD) serve as the additional marker.

19. A method according to claim 18, wherein the concentration of said PD is measured using a redox titration.

20. A method according to claim 19, wherein the titration is iodometric.

21. A method according to claim 18, wherein the concentration of PD is measured using a spectrophotometric assay.

22. A kit for the rapid diagnosis of cardiovascular disease comprising:
a) a receptacle for collection of urine samples;
b) means for measuring the urinary concentration of one or more thromboxanes selected from the group consisting of thromboxane B₂, 11-dehydrothromboxane B₂, 2,3-di-northromboxane B₂, and mixtures thereof;
c) means for measuring the urinary concentration of at least one of Apo(a), CD and PD;
d) means for measuring the conductivity of said urine sample;
e) a reference chart for interpretation of the results obtained in b), c) and d) and for assessing the diagnostic significance of said results; and
f) manufacturer's instructions for use of said kit.

23. A kit according to claim 22, wherein the receptacle comprises tubes enabling the measurement of some of the markers of cardiovascular diseases, recited in claim 1, directly in said tubes.

24. A kit according to claim 23, wherein the measurement comprises spectrophotometry, turbidimetry, immunoassays, or titrations.

25. A kit according to claim 24, wherein said means are a dipstick-type device.

26. A kit according to claim 22, wherein said means are reagents for spectrophotometric determination of one or more of the markers of cardiovascular diseases in urine.

27. A kit according to claim 22, wherein said means are reagents for determination of one or more of the markers of cardiovascular diseases in urine using an immunoassay.

28. A kit according to claim 23, wherein the tubes are provided with stoppers, and color-forming reactions of a spectrophotometric assay can be performed directly in said tubes.

29. A kit according to claim 23, wherein the tubes are transparent and for use with a spectrophotometer.

30. A kit according to claim 29, wherein the tubes are adopted for direct reading of absorbance in a spectrophotometric assay.

31. A kit according to claim 22, wherein said means for measuring the conductivity of said urine sample are a conductometric electrode that is adopted for measuring the conductivity of said urine sample directly in said tube.
